(19) United States
(12) Patent Application Publication
(10) Pub. No.: US 2023/0348859 A1
(43) Pub. Date: Nov. 2, 2023

(54) DECELLULARIZED MAMMALIAN EXTRACELLULAR MATRIX MORSELS, METHODS MAKING AND METHODS OF USING SAME

(71) Applicant: Brown University, Providence, RI (US)

(72) Inventors: Blanche C. IP, Providence, RI (US); Jeffrey R. MORGAN, Providence, RI (US)

(21) Appl. No.: 18/339,980

(22) Filed: Jun. 22, 2023

Related U.S. Application Data
(63) Continuation of application No. PCT/US2021/064834, filed on Dec. 22, 2021.
(60) Provisional application No. 63/129,966, filed on Dec. 23, 2020.

(51) Int. Cl. C12N 5/077

(52) U.S. Cl. CPC C12N 5/0657; C12N 2533/90; C12N 2501/998; C12N 2501/90; C12N 2539/00; C12N 2510/00

(57) ABSTRACT

Described are decellularized human extracellular matrix (ECM) morsels for use in tissue regeneration and repair.

### CROSS-REFERENCE TO RELATED APPLICATIONS

This patent application is a continuation of International PCT Application No. PCT/US2021/064834, filed on Dec. 22, 2021, and published as WO 2022/140530 A1 on Jun. 30, 2022, which claims priority to U.S. provisional patent application 63/129,966, filed Dec. 23, 2020, the contents of which are incorporated herein by reference.

### GOVERNMENT LICENSE RIGHTS

This invention was made with government support under grant number R03 EB028056 awarded by the National Institutes of Health. The government has certain rights in the invention.

### TECHNICAL FIELD

The present disclosure generally relates to the field of tissue regeneration and repair, particularly utilizing decellularized mammalian extracellular matrix (ECM) morsels, such as decellularized human ECM morsels.

### BACKGROUND

Organ failure, such as resulting from disease or trauma, poses substantial health and cost issues to society. For example, successful treatment often requires the repair or replacement of the organ, but an increasing shortage of transplantable organs has resulted in a wait-list of over 100,000 patients in the US alone. The situation is particularly dire for patients with cardiovascular disease; approximately 790,000 Americans suffer a myocardial infarction (MI) each year. While up to 50% of MI patients survive, all will have sustained progressive cardiac tissue damage, and this progressive damage is a leading cause of mortality for MI survivors worldwide. Indeed, many survivors subsequently develop heart failure, for which the 5-year survival rate is only 50%.

No current treatments can prevent post-MI heart failure; heart transplants and left ventricular assist devices are the only options for end-stage heart failure. Both options are expensive and have limitations, including scarcity of donor organs.

The heart possesses regeneration potential derived from endogenous and exogenous stem and progenitor cell populations, though baseline regeneration appears to be sub-therapeutic. This limitation was initially attributed to a lack of cells with cardiomyogenic potential following an insult to the myocardium. However, recent studies demonstrate increased numbers of cardiomyocyte progenitor cells in diseased hearts. Given that the limiting factor does not appear to be cell quantity but rather repletion of functional cardiomyocytes, it is crucial to understand potential mechanisms inhibiting progenitor cell differentiation. An area of interest in heart disease treatment is extracellular matrix (ECM) remodeling, with both the composition and mechanical properties of the ECM undergoing changes in diseased hearts.

Alternative treatments utilizing tissue engineering could help bridge the gap between available organs and patients in need of new organs to survive. For example, a natural scaffold, ECM, can support tissue repair and regeneration. Decellularized organ tissue from animals can be processed into an injectable liquid that solidifies into a gel at the site of injection. However, the ECM from animals is composed of biomolecules and protein sequences foreign to humans that can elicit an immune reaction that limits effectiveness or even causes further tissue damage. At the same time, it has been challenging to extract ECM from human-derived tissues, and various pathologies and post-mortem degradation of human tissue also render them unacceptable ECM material for tissue repair.

Accordingly, there is a need for novel sources and methods of preparing decellularized mammalian, for example human, ECM morsels (spheroid-shaped microtissues) for use in tissue repair and regeneration.

### SUMMARY

The present disclosure, in part, relates to novel sources, compositions, and methods of preparing and using decellularized mammalian ECM particles or morsels. Generally, the methods and compositions disclosed herein relate to the use of human ECM for the purpose of tissue repair and regeneration, for example in therapeutic or cosmetic procedures.

Disclosed embodiments comprise three-dimensional (3D) in-vitro cell culture systems to engineer specifically-tailored microtissues suitable for particular patients and indications. In embodiments, the three-dimensional (3D) in-vitro cell culture systems comprise scaffold-free systems.

Disclosed tissues can be decellularized to fabricate decellularized mammalian extracellular matrix (ECM), for example in the form of small porous particles or morsels that are equal to or less than 800 um in diameter, thus providing “flowable” compositions that can be administered with, for example, a cannula such as a needle. Disclosed compositions can be administered via injection due to the size and shape of the ECM particles/morsels.

The resulting decellularized mammalian ECM particles/morsels can have a number of clinical applications including but not limited to supporting tissue regeneration.

Disclosed herein are customized decellularized mammalian ECM compositions. For example, in embodiments, disclosed compositions are derived from cultured human cells. In embodiments the human cells can comprise heart or lung cells. In embodiments, the human cells can comprise recombinant human cells.

Disclosed herein are methods of producing customized decellularized ECM compositions, for example mammalian ECM compositions. For example, in embodiments, disclosed methods comprise production of ECM derived from cultured human cells. In embodiments the human cells can comprise heart or lung cells. In embodiments, the human cells can comprise recombinant human cells. Disclosed methods provide for faster, more efficient decellularization as compared to methods previously known in the art.

Disclosed herein are methods of using customized decellularized mammalian ECM compositions. For example, in embodiments, disclosed methods of use comprise, for example, repair and regeneration of, for example, a cardiovascular injury in, for example, a mammal such as a human. In embodiments, disclosed methods of use can comprise administration of a disclosed ECM composition to a treatment area, for example the heart. In embodiments, disclosed compositions can be administered via injection, as a liquid such as an aerosol, or as an impregnated patch. In embodiments, disclosed flowable compositions can be administered using a cannula such as a needle, for example a syringe.

### BRIEF DESCRIPTION OF THE DRAWINGS

FIGS. 1A-1B illustrate the ECM (FIG. 1A), a fibrous network of proteins, proteoglycans, and glycoproteins arranged in a three-dimensional (3D) architecture, and its uses in human tissue regeneration after damage, such as a myocardial infarction (FIG. 1B).

FIGS. 2A-2D illustrate a method of generating spheroid-shaped microtissues for fabricating human ECM morsels. The process begins by seeding cultured human cells into micro-wells using a non-adhesive micro-mold platform technology (FIGS. 2A and 2B). See, e.g., U.S. Pat. No. 8,361,781 (Morgan et al.), herein incorporated by reference in its entirety. The seeded cells aggregate, synthesize, assemble, and deposit human ECM. Resulting ECM microtissues or spheroids (each approximately 50 to 300 μm in diameter) within each of the micro-wells (each approximately 400 — 800 μm in diameter) are illustrated in FIGS. 2C and 2D.

FIGS. 3A-3B illustrate disclosed non-adhesive micro-mold platform technology, including schematics of a master mold (FIG. 3A), as well as the corresponding 3D printed mold, the silicone negative, and an image of an agarose mold with ECM microtissue within the micro-wells. FIG. 3B illustrates another embodiment comprising ring- and honeycomb-shaped molds, the corresponding negative replicates in agarose, and the resulting ring- and honeycomb-shaped 3D human ECM microtissues (6 million cells, 2 cm across), removed from the agarose gel and stained for viable cells with calcein-AM. Also illustrated is a decellularized honeycomb-shaped 3D tissue of human dermal fibroblasts (14 million cells) showing ECM made after only 3 days; 2 cm across.

FIG. 4 illustrates a method of generating decellularized human ECM morsels. In FIG. 4 fetal and adult human heart cells or adult lung cells can be cultured and seeded into micro-wells to generate ECM microtissue or spheroids. The ECM spheroid microtissues can be decellularized inside the micro-wells. The resulting decellularized fetal and adult human heart or human lung ECM morsels maintain their spheroid shape following the decellularization procedure. Scale bar=200 μm.

FIG. 5 illustrates an additional or alternative method of generating decellularized human ECM morsels. In FIG. 5 fetal and adult human heart cells can be cultured and seeded into micro-wells to generate ECM microtissue or spheroids. The ECM spheroid microtissues can be collected, decellularized, and mixed, for example through vortexing. The resulting decellularized fetal and adult human heart ECM morsels maintain their spheroid shape following the decellularization procedure.

FIGS. 6A-6B illustrate decellularized human fetal ECM morsels before and after passage through a 27 G syringe 10 times (FIG. 6A), or imaged with an inverted microscope in brightfield with a 10× objective before and after passage through a 27 G syringe one time (FIG. 6B).

FIG. 7A illustrates one embodiment, showing stained ECM spheroid microtissues from human lung cells, either from healthy cells or cells from a patient with idiopathic pulmonary fibrosis (IPF), either untreated or treated with transforming growth factor beta 1, and FIG. 7B illustrates ECM spheroid microtissues from adult and fetal human heart cells (fibroblasts alone, or co-cultured with cardiac myocytes and microvascular endothelial cells). Fibroblast-only adult or fetal heart ECM microtissues were cultured for 3, 6, 9 or 12 days, whereas the tri-cultures were only cultured for 6 days. Images of the ECM spheroid microtissues in brightfield, stained with hematoxylin and eosin (H&E) or SIRIUS RED™, before decellularization are illustrated and ECM spheroid microtissues from adult and fetal human heart or adult lung cells after decellularization are also illustrated. Scale bars=50 μm.

FIG. 8 illustrates that the size increase of human fetal heart microtissues from day 3 to 12 is associated with increases in collagen (μg) and sulphated glycosaminoglycans (sGAG) (μg) content, but not with changes in DNA content (ng/mL).

FIGS. 9A-9C shows lung and heart ECM microtissues under multi-photon confocal second-harmonic generation (SHG) microscopy to capture fibrillar collagen architecture in three dimension. Human adult healthy lung ECM microtissues (FIG. 9A) reveal different fibrillar collagen structure than human adult fibrotic lung ECM (FIG. 9B) and human adult healthy heart ECM microtissues (FIG. 9C).

FIG. 10 Mechanical stiffness of cultured human fetal heart ECM is comparable to healthy human heart tissue. Graphical representation of cultured human ECM elastic moduli (mean±SD kPa) of adult lung (L-A), adult heart (H-A) and fetal heart (H-F), as compared to the stiffness range for healthy human heart (shaded in red), and the equivalent elastic modulus of gelled collagen (Col), Matrigel (Mat), not-crosslinked porcine cardiac ECM (Pig—N) and crosslinked porcine cardiac ECM (Pig—C) as reported in published work from another laboratory. N=9/group.

FIG. 11 shows that cultured ECM is biocompatible in vitro. Representative images of fixed and embedded sections of human cardiac myocyte (HCM) or microvascular endothelial cells (HCMEC) seeded into microwell with or without cultured human adult or fetal heart ECM that were incubated with EdU (5-ethynyl-2′-deoxyuridine) 24 h post cell-seeding for 48 h (HCM) or 24 h (HCMEC) prior fixation. Sections were stained with H&E or Click-iT™ EdU Cell Proliferation Kit, where proliferating cells were labelled with Alexa Fluor™ 488 dye (red) and all nuclei were counterstained in Hoechst 33342 (blue). N=3.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

“Administration,” or “to administer” means the step of giving (i.e. administering) a medical device, material or agent to a subject. The materials disclosed herein can be administered via a number of appropriate routes, but are typically employed in connection with a surgical procedure.

As used herein in the specification and the appended claims, the singular forms “a,” “an,” and “the” include plural referents unless the context clearly dictates otherwise. Thus, for example, reference to “a component” or “the component” includes two or more components.

The term “and/or” used in the context of “X and/or Y” should be interpreted as “X,” or “Y,” or “X and Y.” Similarly, “at least one of X or Y” should be interpreted as “X,” or “Y,” or “X and Y.”

“ECM physical property” refers to properties including but not limited to the shape, size, surface roughness, porosity, fibrillar collagen two-dimensional architecture, fibrillar collagen three-dimensional architecture, of the ECM morsels/particles.

““ECM biochemical property” refers to properties including but not limited to species (identity) and contents (relative amounts) of biochemical molecules (amino acids, peptides, proteins, modified proteins, carbohydrates, fatty acids, glycosaminoglycans, enzymes, signalling molecules (such as transforming growth factor beta 1), cytokines, hormones), as well as the degradability and biocompatibility of ECM morsels/particles.

“ECM mechanical property” refers to properties including but not limited to tensile strength, compressive strength, elastic modulus, shear modulus of ECM morsels/particles.

“In-vivo ECM properties” refers to physical, biochemical, or mechanical properties associated with naturally-occurring ECM.

“Customized ECM” refers to ECM with physical, biochemical, or mechanical properties that differ from those associated with naturally-occurring ECM as, such difference a result of the disclosed methods.

“ECM microtissue” refers to 3D compositions comprising cells and ECM.

“ECM morsels” or “ECM particles” refers to decellularized ECM microtissue.

Ranges can be expressed herein as from “about” or “approximately” one particular value, and/or “about” or “approximately” to another particular value. When such a range is expressed, another embodiment includes from the one particular value and/or to the other particular value. Similarly, when values are expressed as approximations, by use of the antecedent “about” or “approximately,” it will be understood that the particular value forms another embodiment.

The terms “subject” and “patient” are used interchangeably and refer to any individual who is the target of administration or treatment. The “subject” can be a vertebrate, such as a mammal. The “subject” can be a human or veterinary patient. The term “patient” generally refers to a “subject” under the treatment of a clinician, e.g., physician, or a healthcare professional.

The terms “peptide,” “polypeptide,” and “protein” are used interchangeably to refer to a polymer of amino acid residues.

The term “therapeutically effective” refers to the amount of the composition used that is of sufficient quantity to ameliorate one or more causes or symptoms of a disease or disorder. Such amelioration only requires a reduction or alteration, not necessarily elimination. In addition, the term “therapeutically effective” includes the amount of the composition used is of sufficient quantity to initiate and/or support the body's tissue or organ repair processes.

The term “treatment” refers to the medical management of a patient with the intent to cure, ameliorate, stabilize, prevent a disease, pathological condition, or disorder. In addition, the term “treatment” refers to the medical management of a patient with the intent to repair, regenerate, or provide support for the body's repair or regenerative processes, for an injury, tissue damage, or organ damage. This term includes active treatment, that is, treatment directed specifically toward the improvement of a disease, injury, damage, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, injury, or disorder; and supportive treatment, that is, treatment employed to supplement another specific therapy directed toward the improvement of the associated disease, pathological condition, injury, or disorder.

The term “administration” to a subject includes any route of introducing or delivering to a subject an agent. “Administration” can be carried out by any suitable route, including, but not limited to oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like.

The terms “treat,” “treating,” “treatment,” and grammatical variations thereof as used herein, include the administration of a composition with the intent or purpose of partially or completely preventing, delaying, curing, healing, repairing, regenerating, alleviating, relieving, altering, remedying, ameliorating, improving, stabilizing, mitigating, and/or reducing the intensity or frequency of one or more diseases or conditions, a symptom of a disease or condition, or an underlying cause of a disease or condition. Treatments according to the invention may be applied preventively, prophylactically, palliatively or remedially.

### EMBODIMENTS

Various non-exhaustive, non-limiting aspects of compositions according to the present disclosure may be useful alone or in combination with one or more other aspects described herein. Disclosed systems, compositions and methods provide unique advantages to both patients and practitioners. For example, disclosed embodiments can produce customized 3D microtissues specifically tailored for use with a particular patient and/or for a particular treatment. In embodiments, these microtissues can be used to fabricate decellularized mammalian ECM particles/morsels from many different types and combinations of mammalian cells, including and not limited to lung fibroblasts, dermal fibroblasts, cardiac fibroblasts, cardiac microvascular endothelial cells, cardiac myocytes of different ages (adult, fetal, juvenile, etc.). Thus, provided herein are methods for producing customizable ECM in-vitro, including ECM that cannot be made from in-vivo tissues.

The biochemical/chemical composition (both biochemical/chemical species and/or their contents), collagen architecture, and mechanical properties of the ECM particles/morsels generated by the mammalian cells in microtissues are uniquely dependent on the types of mammalian cells used (tissue origin, age, disease state, etc.). Thus, particular cells can be employed to achieve desired ECM properties.

The biochemical/chemical composition (both biochemical/chemical species and/or their contents), collagen architecture, and mechanical properties of the ECM particles/morsels generated by the mammalian cells in microtissues are further dependent on the culturing conditions of the mammalian cells as they form microtissues. For example, cell culture media composition, culturing time, oxygen level, and the presence or amount of additional biological factors including and not limited to growth factors, cytokines, drugs, and the like can be adjusted to produce the desired ECM properties.

The biochemical/chemical composition (both biochemical/chemical species and/or their contents), collagen architecture, and mechanical properties of the disclosed ECM particles/morsels generated by the mammalian cells in microtissues can be different from ECM extracted from mammalian tissues found in nature (for example, pig heart, human heart, etc.).

Disclosed ECM particles/morsels can form flowable compositions that can pass through a syringe with an attached needle, where the needle inner diameter (ID) would depend on the size of the ECM particles/morsels. For example, ECM particles/morsels with a diameter of ˜200 um will be able to pass through any needle with an ID equal or larger than the ID of a 27 G needle (ID=210 um).

Disclosed ECM particles can be made using fewer steps than ECM extracted from animal or human tissues and organs. Disclosed ECM particles can be made using aseptic conditions.

Disclosed decellularized mammalian ECM particles/morsels are biocompatible in-vitro, such that mammalian cells placed on the decellularized mammalian ECM particles/morsels can survive and multiply.

Different ECM characteristics (including, for example, physical properties, biochemical/chemical compositions, collagen architecture, mechanical properties, and combinations thereof) are likely to have different effects on surrounding tissue once implanted in-vivo, allowing for customized ECM to be developed for any number of clinical conditions.

### ECM Compositions

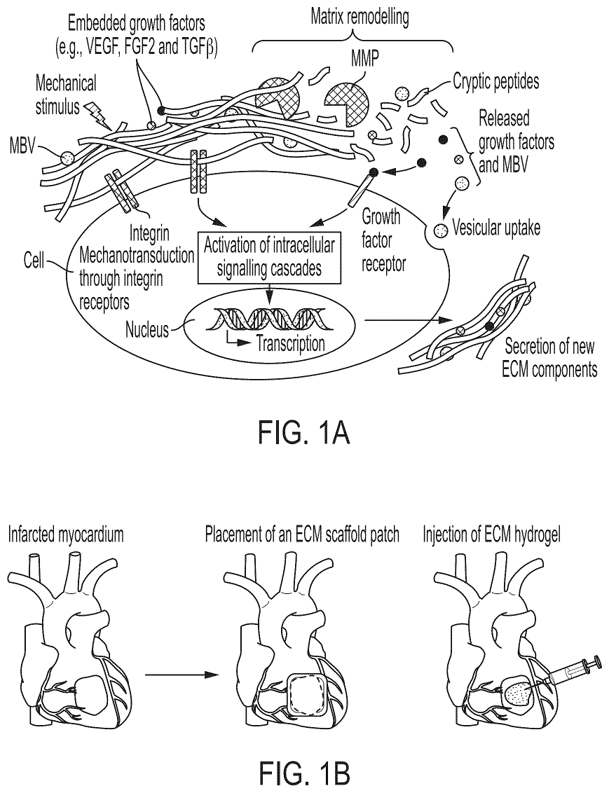

Disclosed herein are customized decellularized compositions, for example decellularized mammalian ECM compositions, whose properties can be specifically tailored to suit particular patients (or patient groups) as well as particular indications. FIG. 1 illustrates the ECM (FIG. 1A), a fibrous network of proteins, proteoglycans, and glycoproteins arranged in a three-dimensional (3D) architecture, and its uses in human tissue regeneration after damage, such as a myocardial infarction (FIG. 1B). The use of foreign, non-human decellularized ECM for tissue regeneration and/or repair can be prone to causing immune reactions in the subject. Decellularized human ECM morsels overcome the issue of immune reactions because the ECM is human. Thus, in embodiments, the decellularized cell are human cells. In embodiments, the human cells can comprise heart or lung cells. In embodiments, the human cells can comprise recombinant human cells. In embodiments, the human cells can comprise at least one of cardiac fibroblasts, cardiac myocytes and cardiac microvascular endothelial cells.

In embodiments, disclosed compositions are derived from cultured human cells used to form decellularized human ECM morsels. The ECM can comprise a complex 3D architecture of structural proteins such as collagen and elastin, along with proteoglycans, enzymes and growth factors (FIG. 1A). In embodiments, the ECM provides structural support, as well as signals for tissue regeneration (FIG. 1B).

### ECM Compositions—Methods of Production

Disclosed herein are methods of producing customized decellularized mammalian-derived ECM compositions with desired physical or chemical properties. For example, in embodiments, disclosed methods are derived from cultured human cells.

Disclosed methods can comprise:
1. Seeding cultured cells (such as mammalian cells) into micro-wells, wherein the cultured mammalian cells generate 3D microtissues, where each of the microtissues are comprised of the cultured mammalian cells and the cell-secreted soluble and insoluble ECM;
2. Collecting microtissues; and
3. Decellularizing microtissues to form ECM morsels or particles with steps that involve in combining the microtissues with detergent, a buffer, a DNase, and an RNase, and mixing/vortexing the mixture.

Various human cell lines can be utilized as sources for disclosed decellularized human ECM morsels. In embodiments the human cells can comprise, for example, heart or lung cells. In embodiments, the human cells can comprise recombinant human cells. For example, in embodiments, human cell lines utilized as sources can include cardiac fibroblasts, cardiac myocytes, cardiac microvascular endothelial cells, and lung fibroblasts. In addition, other human cell types and of different maturation could be used as a source for the ECM morsels. In embodiments, the human cell lines used as the source of the decellularized ECM morsels can be of different maturity, such as adult, juvenile, or fetal cells.

In embodiments, methods of making human ECM microtissues or spheroids are provided. In embodiments, disclosed methods comprise seeding cultured mammalian, for example human, cells into, for example, micro-wells using a non cell-adhesive micro-mold platform technology.

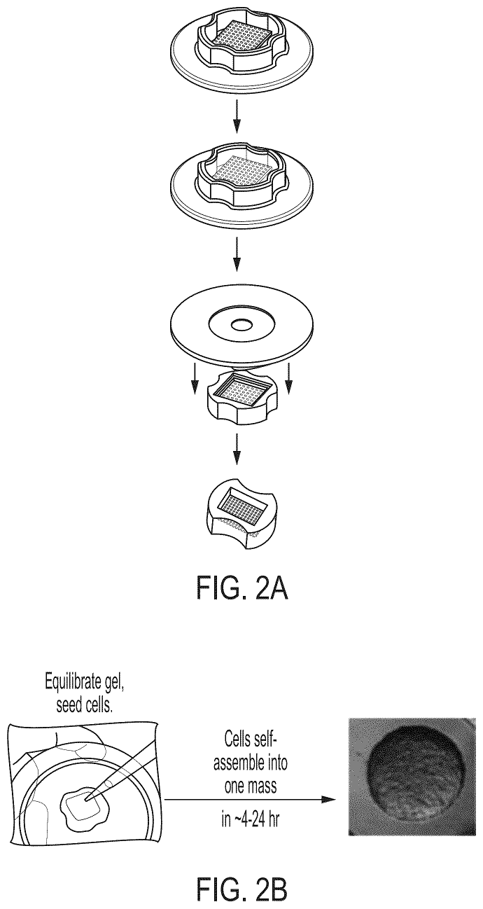

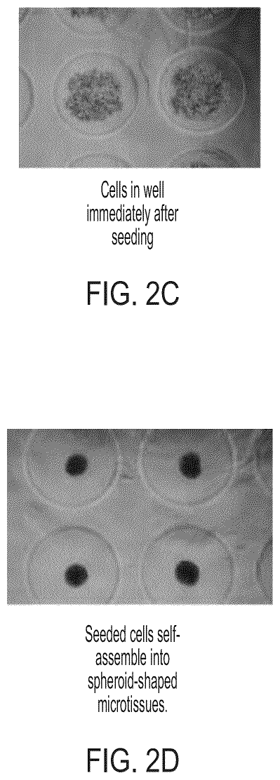

FIG. 2 illustrates a disclosed method for generating spheroid-shaped microtissues for fabricating human ECM morsels. The process begins by seeding cultured human cells into micro-wells using a non-adhesive micro-mold platform technology (FIGS. 2A and 2B). The seeded cells aggregate, synthesize, assemble, and deposit human ECM. Resulting ECM microtissues or spheroids (each approximately 50 to 300 μm in diameter) within each of the micro-wells (each approximately 400-800 μm in diameter) are illustrated in FIGS. 2C and 2D.

In embodiments, the micro-wells can be generated from any suitable material, such as agarose. For example, 2% agarose can be used to generate the micro-wells where the cells, such as living human cells, can aggregate synthesize, assemble, and deposit human ECM.

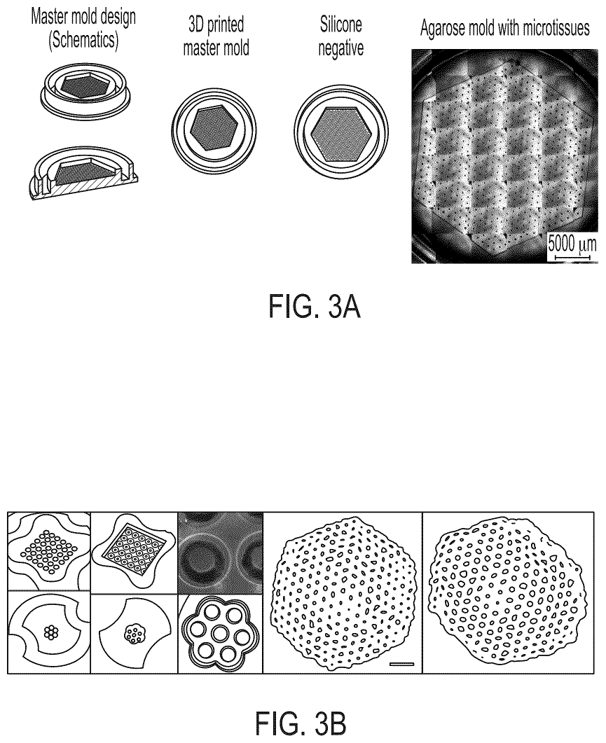

FIG. 3 illustrates an embodiment employing non-adhesive micro-mold platform technology, including schematics of a master mold (FIG. 3A), as well as the corresponding 3D printed mold, the silicone negative, and an image of an agarose mold with ECM microtissue within the micro-wells. FIG. 3B illustrates another embodiment, using ring- and honeycomb-shaped molds, the corresponding negative replicates in agarose, and the resulting ring- and honeycomb-shaped 3D human ECM microtissues (6 million cells, 2 cm across), removed from the agarose gel and stained for viable cells with calcein-AM. Also illustrated is a decellularized honeycomb-shaped 3D tissue of human dermal fibroblasts (14 million cells) showing ECM made after only 3 days; 2 cm across.

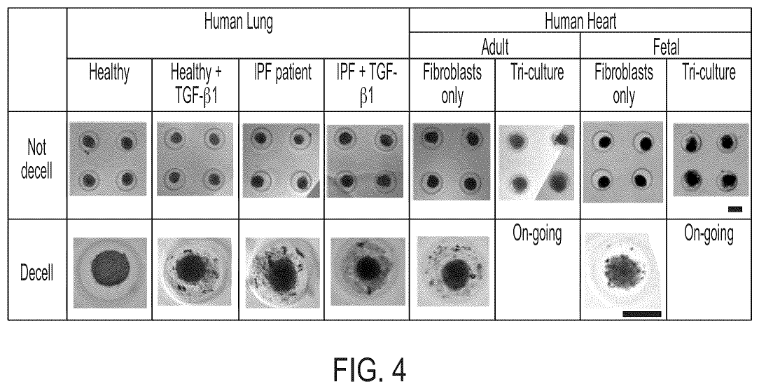

FIG. 4 illustrates a further method of generating decellularized human ECM morsels. In the embodiment of FIG. 4, fetal and adult human heart cells or adult lung cells are cultured and seeded into micro-wells to generate ECM microtissue or spheroids. The ECM spheroid microtissues can be decellularized inside the micro-wells. The resulting decellularized fetal and adult human heart or human lung ECM morsels maintain their spheroid shape following the decellularization procedure. Scale bar=200 μm.

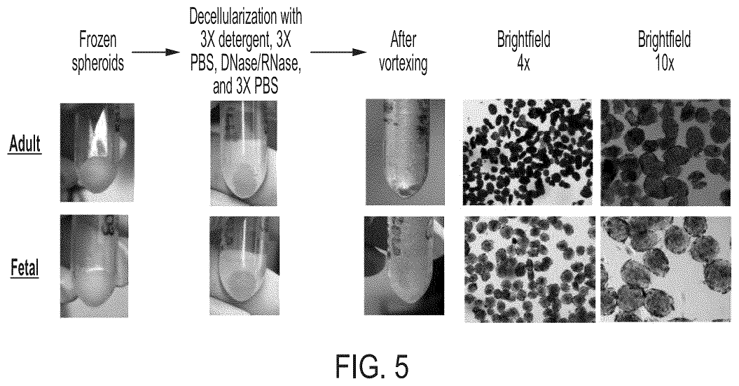

FIG. 5 illustrates an additional or alternative method of generating decellularized human ECM morsels. In FIG. 5, fetal and adult human heart cells can be cultured and seeded into micro-wells to generate ECM microtissue or spheroids. The ECM spheroid microtissues can be collected, decellularized, and mixed, for example vortexed. The resulting decellularized fetal and adult human heart ECM morsels maintain their spheroid shape following the decellularization procedure.

In embodiments, micro-molded, non-adhesive, cell aggregation devices can comprise a plurality of cell aggregation recesses in the shape of, for example, depressions or troughs. In embodiments, agarose can be employed as the hydrogel material and the cell aggregation recesses can be established, in embodiments, as follows. Troughs can be 400 μm wide with bottoms rounded with, for example, 200 μm radii. Disclosed embodiments can comprise rows of troughs of increasing length per gel. For example, in an embodiment, each row can have 11 troughs, two of which are 400 μm long, then one each of 600 μm through 1800 μm increasing at 200 μm lengths, then two 2200 μm troughs. In embodiments, tori-shaped recesses can be 800 μm deep, with circular track 400 μm wide. In embodiments, the recess bottom can comprise a radius of 200 μm.

In embodiments, it can take, for example, about 4 hours to about 24 hours for the cells to assemble into mammalian, for example human, ECM microtissues or spheroids and begin to generate the ECM (FIG. 2C). For example, in embodiments, cell assembly can take 4 hours, 6 hours, 8 hours, 10 hours, 12 hours, 14 hours, 16 hours, 18 hours, 20 hours, 22 hours, 24 hours, 26 hours, 28 hours, or the like.

In embodiments, cell assembly can take at least 4 hours, at least 6 hours, at least 8 hours, at least 10 hours, at least 12 hours, at least 14 hours, at least 16 hours, at least 18 hours, at least 20 hours, at least 22 hours, at least 24 hours, at least 26 hours, at least 28 hours, or the like.

Disclosed methods of generating the mammalian, for example human ECM microtissues or spheroids using the micro-mold technology provides for a stable, long-term, reproducible culture platform to form 3D human ECM microtissues or spheroids at high cell density (FIG. 2C). In addition, the micro-mold technology does not require that a scaffold material be used, thus, in embodiments, only the cultured cells, for example human cells, are needed to add to the micro-wells to generate spheroid-shaped microtissues. This approach allows for optimal cell-to-cell communication and movement, allowing mixtures of different cell types to interact while undergoing complex 3D morphological changes and differentiation. The micro-mold technology can allow cells in micro-wells to be cultured statically with exchange of cell culture medium that allows cell-secreted ECM to be concentrated at the site of secretion. In addition, the micro-molds can be customizable for a wide variety of tissue shapes and sizes based on by initial mold geometry (FIGS. 3A and 3B). Moreover, the mold geometry directs cellular alignment and organization, which subsequently affects the ECM microstructure and alignment, as well as bulk mechanical properties. Thus, unlike scaffold-based or cell-sheet methods, the micro-mold technology can be used to promote ECM alignment to better mimic the native tissue, which can increase therapeutic efficacy by promoting cell attachment and migration. The amount of time required for the seeded cells to sufficiently generate human ECM can be dependent on the type of cell, as well as the growing conditions. In one embodiment, seeded human cardiac fibroblasts can generate ECM in about 3 to about 12 days. For example, in embodiments, seeded human cardiac fibroblasts can generate ECM in about 3 days, 4 days, 5 days, 6 days, 7 days, 8 days, 9 days, 10 days, 11 days, 12 days, or the like.

In embodiments, the human ECM microtissues can then be collected and decellularized, or decellularized directly within the micro-molds. Decellularization can be accomplished by treatment with a mild detergent followed by a treatment to remove DNA (FIG. 3). Disclosed ECM microtissues can be collected from the micro-wells by, for example, pipetting, subjected to an optional freeze-thaw step, treatment with salt solutions and a mild detergent, followed by treatment with the enzymes DNase and RNase can be used to remove the DNA and RNA from the human microtissues.

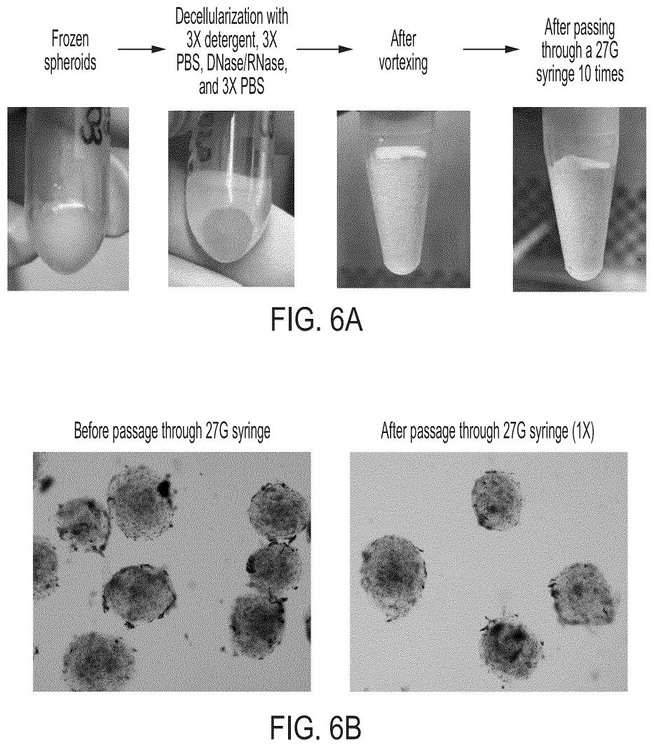

In embodiments, successful decellularization kills the cells, removes most of the cellular material, and removes or destroys most of the DNA, leaving behind human ECM in tissue and age-specific three-dimensional architecture and mechanical stiffness. The resulting composition, comprising decellularized human ECM morsels or small porous spheres of human ECM, can freely pass through a small diameter hypodermic needle (FIG. 4) and can therefore be easily injected into organs or tissues in need of tissue repair or regeneration. Disclosed decellularized human ECM morsels can be evaluated for the presence of DNA using techniques know to a person skilled in the art. DNA-free decellularized ECM can undergo constructive remodeling in-vivo with minimal adverse effects. FIG. 6 illustrates decellularized human fetal ECM morsels before and after passage through a 27 G syringe 10 times.

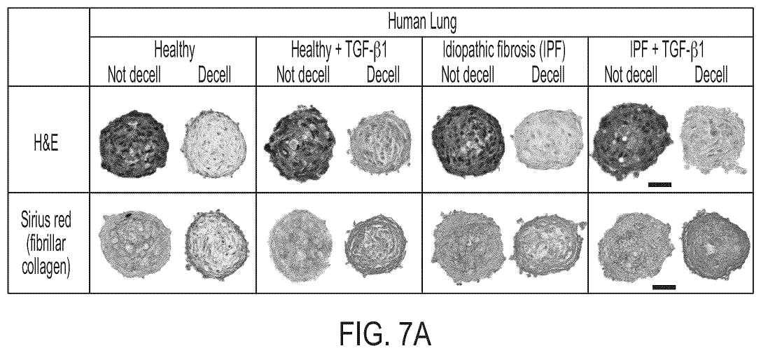

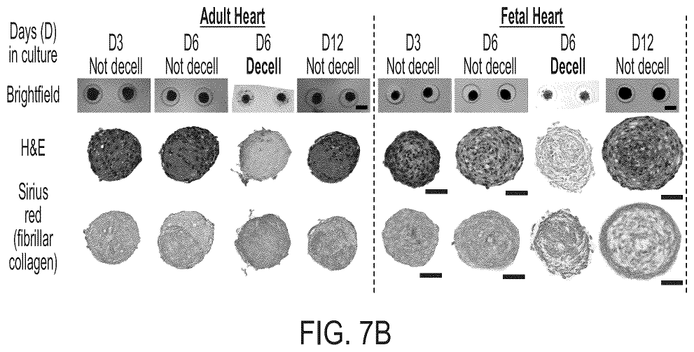

FIG. 7A illustrates one embodiment comprising stained ECM spheroid microtissues from human lung cells, both healthy cells and cells from a patient with idiopathic pulmonary fibrosis (IPF), and FIG. 7B illustrates ECM spheroid microtissues from adult and fetal human heart cells (fibroblasts alone, or co-cultured with cardiac myocytes and microvascular endothelial cells). Fibroblast-only adult or fetal heart ECM microtissues were cultured for 3, 6, 9 or 12 days, whereas the tri-cultures were only cultured for 6 days. Images of the ECM spheroid microtissues in brightfield, stained with hematoxylin and eosin (H&E) or SIRIUS RED™, before decellularization are illustrated and ECM spheroid microtissues from adult and fetal human heart or adult lung cells after decellularization are also illustrated. Scale bars=50 μm.

In accordance with another non-limiting aspect of the present disclosure, which may be used in combination with each or any of the above-mentioned aspects, the composition of the decellularized mammalian, for example human, ECM morsels can be designed for a particular patient in need thereof. In embodiments, the composition of the decellularized human ECM morsels can be generated by selecting a desirable cell type or selecting a combination or mixture of different cell types to generate decellularized human ECM morsels. Starting cell types may include, but are not limited to the following: cardiac fibroblasts, cardiac myocytes and cardiac microvascular endothelial cells could be used in combination to generate a decellularized human ECM that may be administered to a particular patient in need thereof. These “designer” ECM compositions comprise unique compositions and potencies that do not exist in native tissues or whole organs, thus providing the practitioner the ability to design and produce compositions particularly suited for a desired treatment in a specific patient.

In addition to selecting different cell types or combining different cell types to form the starting ECM microtissues, as previously described, “designer” ECM compositions can also be generated by treating the starting 3D ECM microtissues with growth factors and/or drugs that can alter and/or improve the production of ECM and its quality. In some embodiments, additives to the culture media such as growth factors, cytokines and drugs can influence the amounts and types of ECM produced by cells. By incubating the microtissues with anti-inflammatory mediators such as, for example, interleukin 4, interleukin 10, interleukin 11 or interleukin 13 can influence the microtissues to produce an “anti-inflammatory designer” ECM. Further “designer” ECM compositions can comprise ECM produced from recombinant cells, for example recombinant cells producing a cytokine.

Additionally or alternatively, as previously explained, the desired size (i.e., diameter) and/or shape of the decellularized human ECM morsels can be changed or adjusted via the number of cells seeded into the micro-molds, the geometry of the initial micro-mold, or the length of culture time for ECM morsels made with human cells. A particular micro-mold could be used to generate 3D ECM spheroid microtissues of a precise size can generate decellularized human ECM morsels that can pass through the desired need size during administration.

In accordance with another non-limiting aspect of the present disclosure, a method of generating decellularized human ECM morsels from cultured cells can be performed using an automated process.

The presently disclosed methods of generating decellularized human ECM morsels from cultured cells for use in tissue repair and regeneration can result in a purer ECM composition, because the starting material is generally more highly defined with no fat, fascia or connective tissues, bacteria, and/or other materials that can contaminate whole organs. There can also be less batch-to-batch variability of ECM compositions derived from cultured cells rather than whole organs. In addition, the decellularized human ECM morsels derived from cultured cells require fewer steps and a gentler process that can preserve function as compared to decellularized human ECM morsels derived from whole organs or from cultured cell sheets. The disclosed processes for producing decellularized human ECM morsels eliminate the steps of lyophilization, digestion, and reconstitution which are typically more laborious, costly and most importantly, disrupt the structure of ECM and its potency.

### ECM Compositions—Methods of Use

Disclosed herein are methods of using disclosed, customized decellularized human ECM compositions. For example, in embodiments, disclosed methods of use comprise treatment of, for example, heart disease.

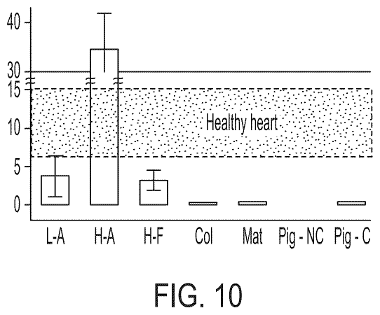

It has been shown that the structural, biochemical and mechanical cues of ECM can facilitate cell attachment, migration and signalling, all of which are critical for tissue regeneration and repair. It is hypothesized that a stiffness similar to healthy myocardium would be ideal for cardiac tissue engineering. FIG. 10 shows that decellularized fetal human heart ECM morsels had comparable mechanical stiffness (elastic modulus) as healthy heart tissue, thus providing an ideal mechanical property for use in heart treatments.

In one aspect, disclosed herein, are methods of using the decellularized human ECM morsels as treatments for the purposes of tissue repair, tissue regeneration, and tissue augmentation. The decellularized human ECM composition may be formulated as an injectable, as a patch, and/or as an aerosol. In embodiments comprising a patch substrate, the patch can comprise a biodegradable material, i.e. it is naturally absorbed by the patient's body after some time. In embodiments, the biodegradable material is biocompatible, i.e. have no harming effect to the patient to whom the material is administered. Thus, the biocompatible matrix can be a biomaterial selected from biopolymers such as a proteins or polysaccharides, for example a biomaterial such as collagen, gelatin, fibrin, a polysaccharide, e.g. hyaluronic acids, chitosan, and derivatives thereof, collagen, chitosan, etc.

In another embodiment, customized decellularized mammalian, for example human, ECM morsels can be injected into the skin to achieve augmentation as a strategy for tissue repair as well as for cosmetic applications and treatments, such as, for example, treatment of the lip, treatment of the cheek, treatment of the forehead, dermal filler treatments, or the like. For example, in an embodiment, a disclosed ECM composition comprising hyaluronic acid can be administered to a subject's lips to add fullness.

Disclosed embodiments can comprise treatment to reduce the effects of aging upon tissues such as skin. For example, over time, various body structures lose function in an unpredictable sequence. ECM provides a commonality amongst these intricate processes, and thus disclosed methods can comprise treatment to reduce the effect of age upon the skin.

Furthermore, in another embodiment, decellularized human ECM morsels can be applied topically to aid in wound healing, for example as a solution, gel, or patch.

In accordance with another non-limiting aspect of the present disclosure, which may be used in combination with the other aspects, compositions of decellularized human ECM morsels can be mixed with stem cells and used as cell carriers for the safe transplantation of administered cells, or mixed with therapeutic compounds/drugs as a delivery agent, such as via injection are described.

Also disclosed herein is the ability of administered decellularized human ECM morsels to promote cell viability, cell proliferation, cell migration, chemotaxis, and/or capillary tube formation in vivo.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

As various changes could be made in the above-described sources and methods without departing from the scope of the invention, it is intended that all matter contained in the above description and in the examples given below, shall be interpreted as illustrative and not in a limiting sense.

### EXAMPLES

### Example 1

An exemplary method of making customized decellularized human ECM morsels is illustrated in Example 1.

### Materials and Methods

### Cell Culture, Micro-Mold Fabrication, and Formation of Microtissues (Single Multi-Cellular Structures)

Human lung fibroblasts (LF, ATCC CRL-4058) were expanded in Fibroblast Basal Medium (ATCC PCS-201-030) supplemented with Fibroblast Growth Kit-Low Serum (ATCC-201-041) and puromycin (Gibco A1113803) at a concentration of 0.3 μg/mL, treated with or without recombinant human transforming growth factor beta 1 (TGF-β1) protein (R&D systems, Minneapolis, MN, 240-B) at 0.625 to 10 ng/mL. Human lung fibroblasts from idiopathic fibrosis patient (IPF, ATCC PCS-201-020) were expanded in Fibroblast Basal Medium (ATCC PCS-201-030) supplemented with Fibroblast Growth Kit-Low Serum (ATCC-201-041), treated with or without recombinant human TGF-β1 protein (R&D systems, 240-B) at 0.625 to 10 ng/m L.

Human lung fibroblasts from patient with chronic obstructive pulmonary disease (COPD, ATCC PCS-201-017) were expanded in Fibroblast Basal Medium (ATCC PCS-201-030) supplemented with Fibroblast Growth Kit-Low Serum (ATCC-201-041). Human cardiac fibroblasts (HCF; Promocell C-12375) were expanded in Fibroblast Growth Medium 3 (C-23025). Human cardiac myocytes (HCM; Promocell C-12810) were expanded in Myocyte Growth Medium 3 (C-22070). Human cardiac microvascular endothelial cells (HCMEC; Promocell C-12285) were expanded in Endothelial Cell Growth Medium MV (C-22020). Human fetal cardiac fibroblasts (HFCF, Cell Applications Inc., San Diego, CA, 306-05f) were expanded in HCF Growth Medium (Cell Applications 316-500). Cells were trypsinized, counted, and re-suspended to the desired cell density for each experiment.

The inventors cast agarose gels from 3D Petri Dish® micro-molds (Microtissues, Inc., Providence, RI, USA) as previously described by Napolitano et al. (2007) Biotechniques 43 (4):494, 496-500. Agarose gels were made with powdered agarose (Low-EEO/Multi-Purpose/Molecular Biology Grade, Fisher BioReagents, Thermo Fisher Scientific) sterilized by autoclaving and then dissolved in sterile phosphate buffered saline (PBS, HyClone SH30256.FS) to 1.5-2% (weight/volume). Micro-molds with round micro-wells were used to create spheroid-shaped microtissues. Round micro-wells for spheroids were 400 to 800 μm in diameter and contained either 35, 96, or 256 micro-wells per gel.

Additionally or alternatively, one of skill in the tissue engineering art could use computer-assisted design (e.g., Solid Works, Concord, MA) to create a template of the desired gel features (e.g., a cell seeding chamber, 721 micro-wells with hemispherical bottoms, 800 μm deep×600 μm wide). Then, one can generate a negative plastic mold with a prototyping machine (e.g., composed of acrylonitrile butadiene styrene (ABS) plastic (Protolabs)). Next, one can fill the negatives (e.g., with silicone rubber compound; MOLDMAX™ 25, Smooth-On, Macungie, PA) to produce positive replicates. The positive replicates are washed (e.g., with 70% ethanol, then rinsed with distilled water) and autoclaved before use. Then, one of ordinary skill in the tissue engineering can cast agarose gel with micro-wells directly from silicone molds, e.g., according to the methods of Napolitano et al. (2007) Biotechniques 43 (4):494, 496-500, whereby 4 mL aliquot of molten 1.5-2% agarose-PBS solution is pipetted into each silicone mold in a sterile environment.

Agarose gels with micro-wells were seeded with trypsinized and counted cells at a density of 500 to 4,000 cells per spheroid micro-well. Cells aggregated in each of the micro-wells within the agarose gels, and self-assembled into 1 spheroid-shaped microtissue per micro-well within 4 to 24 hours after cell-seeding.

Cells within the micro-wells were cultured for 3, 6, 9 or 12 days after cell-seeding in a humidified incubator with 5% CO² and at 37° C., with media change every 3 days.

### Visual Inspection of Microtissues

Microtissues in agarose gels were inspected with inverted light microscopy fitted with camera (e.g. Nikon Ti2, Zeiss Axio Observer Z1 or similar) to examine the size of the microtissues. The cross-sectional area of microtissues were measured using ImageJ (US National Institutes of Health, Bethesda, MD).

### Examine Histology of Microtissues

Microtissues cultured for different days were fixed in 10% buffered formalin (Fisher 427098) in the agarose gels, paraffin-embedded, sectioned at 5 μm then stained with hematoxylin and eosin (H&E) or SIRIUS RED™ (Polyscience, Warrington, PA, 24901-250) to examine microtissue morphology or fibrillar collagen deposition, respectively.

### Examine Fibrillar Collagen Structure Microtissues

Microtissues cultured for different days were fixed in 10% buffered formalin (Fisher 427098) in the agarose gels. Fibrillar collagen was visualized using an Olympus FV-1000-MPE multiphoton microscope (Olympus, Tokyo, Japan) equipped with a Mai Tai HP tunable laser with the excitation wavelength set to 790 nm and a 405/40 filter cube to select for fibrillar collagen second-harmonic signal. Microtissues fixed in 10% buffered formalin imaged in the agarose gel with a 25× (Numerical Aperture 1.05, Working Distance 2 mm) dipping objective in PBS.

### Examine the Proteomics of Microtissues

Microtissues in agarose gels were washed with PBS three times then collected into a tube. ECM-enrichment and proteomics procedures as previously described by Naba et al. (2015) J Vis Exp, 2015 (101): p. e53057 were used to decellularize the microtissue and concentrate ECM proteins for proteomics analysis of the microtissues. Mass spectrometry by data dependent acquisition (DDA) and data analysis with Proteome Discoverer 2.3 (1% FDR) were used for the proteomics analysis of ECM proteins. An established iBAQ algorithm as described by Schwanhausser et al. (2011) Nature, 2011. 473 (7347): p. 337-42 was used to semi-quantity ECM components (by % molar of total ECM proteins) by dividing each individual protein's total intensity with the theoretical number of tryptic peptides between 6 and 30 amino acids in length (PeptideMass, SIB Swiss Institute of Bioinformatics).

### Decellularization of Microtissues

Microtissues in agarose gels were washed with PBS three times. Decellularization of microtissues were either completed with microtissues remaining in the agarose gels, or after microtissues were collected into a tube. Microtissues in gels or in tubes were first treated with three rounds of 0.5% Triton-X100 (MilliporeSigma, St Louis, MO, T9284) in 20 mM NH₄OH (MilliporeSigma, 09859) in sterile PBS with protease inhibitors (PI; ThermoFisher Scientific, PI78439) for 45 mins with 60 rpm rotation per incubation, followed by three rounds of washes with sterile PBS+PI for 45 mins with 60 rpm rotation per incubation, then subjected to 1 round of incubation with DNase I (MilliporeSigma, 4716728001)+RNase A (Qiagen, Hilden, Germany, 19101)+PI for 72 hours with 60 rpm rotation, followed by three rounds of washes with sterile PBS+PI for 45 mins with 60 rpm rotation per incubation. The resulting decellularized microtissue ECM morsels were stored in sterile PBS at 4° C. for visual inspection and mechanical testing, fixed in 10% buffered formalin for histological analysis, or snap-frozen in liquid nitrogen and stored at −80° C. for subsequent biochemical analysis.

### Visual Inspection of Decellularized Microtissue ECM Morsels

Decellularized microtissue ECM morsels in agarose gels were inspected with inverted light microscopy fitted with a camera as previously described to examine the size and architecture of the decellularized microtissue ECM morsels. Decellularized microtissue ECM morsels in tubes in sterile PBS were vortexed then photographed with iPhone, or transferred to a sterile 24-well plate (Corning, NY, 3527) then imaged with inverted light microscopy as previously described.

### Examine Histology of Decellularized Microtissue ECM Morsels

Decellularized microtissue ECM morsels in agarose gels were fixed in 10% buffered formalin (Fisher 427098) in the agarose gels, paraffin-embedded, sectioned at 5 μm then stained with hematoxylin and eosin (H&E) or SIRIUS RED™ (Polyscience, Warrington, PA, 24901-250) to examine the presence of cell nuclei or fibrillar collagen deposition of decellularized microtissue ECM morsels, respectively.

### Examine dsDNA Concentration of Decellularized Microtissue ECM Morsels

DsDNA concentration of decellularized microtissue ECM morsels were measured as previously described by Blaheta et al. (1998) J Immunol Methods 1998 Feb. 1; 211 (1-2):159-69. Decellularized microtissue ECM morsels that were collected into a tube were digested in papain solution (MilliporeSigma, P4762, 125 μg/mL) in a sonicator for 72 hours at 65° C. The dsDNA concentration of the digested ECM was measured using QUANT-IT™ PICOGREEN™ dsDNA Assay Kit (Thermo Fisher Scientific, P7589) per manufacture's protocol.

### Examine Collagen Content of Microtissues

Collagen content of microtissues was measured as previously described by Cissell et. al (2017) Tissue Eng Part C Methods 2017 April; 23 (4):243-250. Microtissues were fixed in 10% formalin and stored at 4° C. until further processed. Fixed microtissues were collected into a tube and washed three times with 1×PBS, then digested in papain solution (MilliporeSigma, P4762, 125 μg/mL) in a sonicator for 10 days at 65° C. The digested microtissues was measured using a modified hydroxyproline assay as described by Cissell et al. (2017).

### Examine Sulphated Glycosaminoglycans (sGAG) Content of Microtissues

sGAG content of microtissues was measured using the 1,9-dimethylmethylene blue (DMMB) assay as described by Farndale et al. (1982) Connect Tissue Res 9 (4):247-248, and Whitley et al. (1989) Clin Chem 35 (3):374-379. Microtissues were fixed in 10% formalin and stored at 4° C. until further processed. Fixed microtissues were collected into a tube and washed three times with 1×PBS, then digested in papain solution (MilliporeSigma, P4762, 125 μg/mL) in a sonicator for 10 days at 65° C. The digested microtissues was measured using the DMMB assay as described by Farndale et al. (1982) and Whitley et al. (1989).

### Examine the Proteomics of Decellularized Microtissue ECM Morsels

Decellularized microtissue ECM morsels that were collected into a tube underwent proteomics procedures as previously described by Naba et al. (2015) J Vis Exp, 2015 (101): p. e53057 for proteomics analysis of the microtissues. Mass spectrometry by data dependent acquisition (DDA) and data analysis with Proteome Discoverer 2.3 (1% FDR) were used for the proteomics analysis of ECM proteins. An established iBAQ algorithm as described by Schwanhausser et al. (2011) Nature, 2011. 473 (7347): p. 337-42 was used to semi-quantity ECM components (by % molar of total ECM proteins) by dividing each individual protein's total intensity with the theoretical number of tryptic peptides between 6 and 30 amino acids in length (PeptideMass, SIB Swiss Institute of Bioinformatics).

### Examine Mechanical Stiffness of ECM Morsels

Samples tested went through a decellularization process, where plated on collagen-coated coverslips and incubated on the coverslips at 4° C. for 48 hrs prior to testing. Force measurements were collected using an atomic force microscope (AFM, MFP-3D-BIO, Asylum Research, Santa Barbara, CA) connected to a Nikon Eclipse Ti-U epifluorescence microscope (Nikon, Chicago, IL). The cantilever used had a spring constant of 0.03 N/m. Multiple testing sessions were conducted for the various samples to account for systematic errors. Force versus indentation data were analyzed using custom MATLAB scripts (The MathWorks, Natick, MA) utilizing the Hertz contact model.

All experiments were carried out at room temperature in fluid environments. The AFM was allowed to equilibrate before tests to minimize deflection laser and/or piezo drift. Force maps were collected for a variety of samples using a force mapping technique in contact mode. In brief, individual force curves were taken at discrete points across a region of interest. During analysis, the spatial arrangement of the data was retained to create a matrix of elastic modulus values. Force—indentation data were sampled at 5 kHz with an approach velocity of 10 μm/sec. A trigger force of about 4 nN was used for all samples with the deflection set to 100 nM. Scan size used was 5 μm and the resolution was 4×4 pts.

### Test Injectability of ECM Morsels

Fetal cardiac microtissues were collected in a single tube and decellularized. The decellularized cultured fetal heart ECM in tube was imaged with a camera after vortexing (FIG. 6A). The cultured fetal heart ECM in tube were then passed through a 27-gauge syringe (inner diameter 0.21 mm) 10 times, then imaged the cultured ECM in tube again after syringe passage.

Some of the cultured fetal heart ECM were transferred with a sterile transfer pipet (with wide opening) into a clean well in 24-well plate lined with a thin layer of 2% (w/v) agarose for imaging under a Nikon microscope with a 10× objective (FIG. 6B). To mimic the surgical cultured ECM injection process, we removed the plunger of a 1 mL sterile syringe then transferred the cultured fetal heart ECM with a sterile transfer pipet into the open end of a 1 mL syringe connected to a 27-gauge needle. The syringe plunger was then placed back onto the opening end of the syringe containing the cultured ECM. Culture fetal heart ECM was then passed through a 27-gauge needle once directly into an unused well in the 24-well plate lined with a thin layer of 2% (w/v) agarose for imaging under a Nikon microscope with a 10× objective (FIG. 6B).

### Examine in Vitro Biocompatibility of ECM Morsels

Decellularized adult or fetal heart ECM morsels were seeded with HCM or HCMEC and incubated for 24 hours. To examine proliferation, the nucleoside analog EdU (5-ethynyl-2′-deoxyuridine) was added 24 hours after cell seeding. Cells on ECM morsels in EdU were cultured for another 24 hours (HCMEC) or 48 hours (HCM), then fixed in 10% formalin for immunohistochemical evaluation using the Click-iT™ EdU Cell Proliferation Kit (Thermo Fisher Scientific) according to the manufacturer's protocol.

### Example 2

### Results

Spheroid-shaped ECM microtissues made with different human cells deposit ECM.

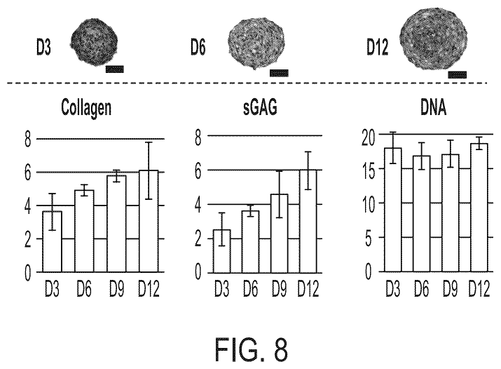

Eight different types of human microtissues were generated from human lung fibroblasts (LF) with or without TGF-β1, fibrotic human lung fibroblasts (IPF) with or without TGF-β1, COPD human lung fibroblast (COPD), adult human cardiac fibroblasts (HCF), fetal human cardiac fibroblasts (HFCF), tri-culture of HCF with human cardiac myocytes (HCM) and human cardiac microvascular endothelial cells (HCMEC), and tri-culture of HFCF, HCM and HCMEC as previously described. FIG. 4 illustrates eight out of nine different types of ECM microtissues in brightfield six days after seeding, and FIG. 7 illustrates microtissue sections stained with H&E and SIRIUS RED™ for the presence of fibrillar collagen (a common type of ECM) in all eight ECM microtissues. FIG. 7B and FIG. 8 illustrate that human fetal heart but not adult heart microtissues grew in size, had increased fibrillar collagen deposition and exhibited greater fibrillar collagen remodeling over culturing time.

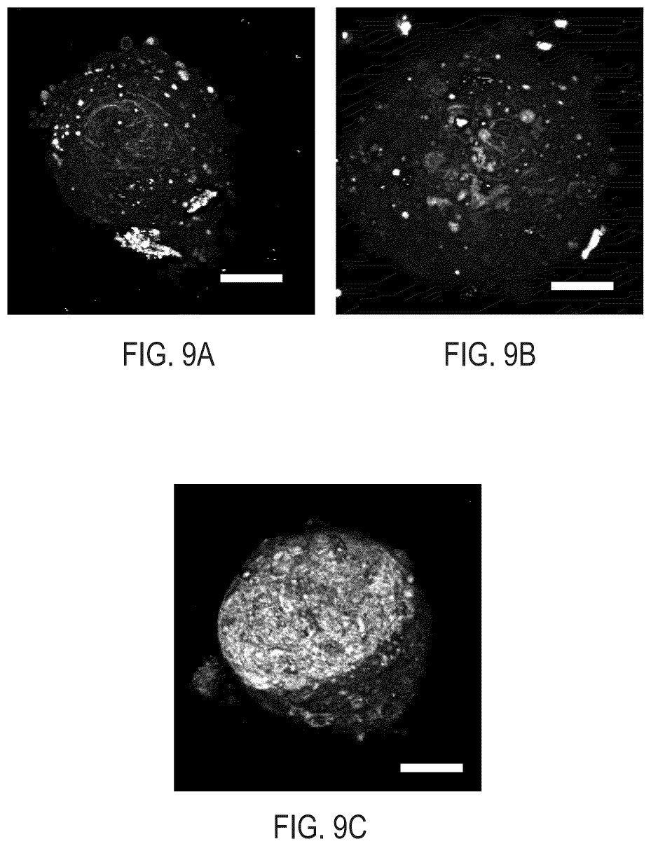

FIG. 9 shows human adult healthy lung ECM microtissues (FIG. 9A) had different fibrillar collagen structure than human fibrotic lung (FIG. 9B) and human adult healthy heart ECM microtissues (FIG. 9C) under multi-photon confocal second-harmonic generation (SHG) microscopy.

### Proteomics Analysis Showed That ECM Protein Compositions Microtissues Were Dependent on Cell Types Used to Make the Microtissues

After ECM-enrichment of microtissues, proteomics was performed to analyze the ECM composition of the various human ECM microtissues. Table 1 displays the percentage of each class of proteins for seven of the nine types of human ECM microtissues.

TABLE 1
Proteome Analysis of Seven Different Human ECM microtissues

| | | | | Healthy | | Healthy | |
|---|---|---|---|---|---|---|---|
| | Healthy | Fibrotic | COPD | Adult Heart - | Healthy | Fetal Heart - | Healthy |
| | Adult | Adult | Adult | Fibroblasts | Adult Heart - | Fibroblasts | Fetal Heart - |
| Protein | Lung | Lung | Lung | only | Triculture | only | Triculture |
| Class | (%) | (%) | (%) | (%) | (%) | (%) | (%) |
| Collagen | 29.59 | 9.93 | 17.39 | 3.99 | 2.90 | 7.53 | 4.30 |
| ECM | 2.46 | 4.46 | 5.11 | 6.49 | 7.02 | 6.28 | 7.13 |
| Regulators | | | | | | | |
| ECM-Affiliated | 15.44 | 24.43 | 22.42 | 21.86 | 26.93 | 18.24 | 28.18 |
| Proteins | | | | | | | |
| Glycoproteins | 49.06 | 48.67 | 41.73 | 40.42 | 35.14 | 58.38 | 40.38 |
| Proteoglycans | 0.42 | 0.96 | 1.79 | 3.65 | 2.77 | 2.05 | 1.29 |
| Secreted | 3.04 | 11.56 | 11.56 | 23.59 | 25.24 | 7.52 | 18.72 |
| Factors | | | | | | | |

The collagen and sGAG content of human ECM microtissues are tissue and age specific. After digestion of ECM microtissues, collagen and sGAG contents were evaluated using a modified hydroxyproline assay and the DMMB assay, respectively. Table 2 displays the collagen and sGAG content in microgram per one million cells of spheroid microtissues for five of the eight types of human ECM microtissues.

TABLE 2
Collagen and sGAG content (μg/10⁶ cells) of Five
Different Human ECM microtissues.

| | Collagen (μg) | sGAG (μg) |
|---|---|---|
| Healthy Adult Lung | 6.35 ± 2.42 | 2.83 ± 0.50 |
| Healthy Adult Lung + TGFβ treatment | 4.84 ± 0.82 | 4.81 ± 0.09 |
| Fibrotic Adult Lung | 2.87 ± 0.51 | 5.11 ± 1.68 |
| Healthy Adult Heart | 2.20 ± 0.17 | 6.16 ± 0.42 |
| Healthy Fetal Heart | 3.63 ± 1.68 | 4.79 ± 1.47 |

Spheroid-shaped microtissues can be decellularized in the agarose gel with micro-wells to efficiently remove cell nuclei while retaining ECM in morsels geometry. Six different types of decellularized human ECM morsels were generated from human LF with or without TGF-β1, IPF with or without TGF-β1, HCF, and HFCF as previously described. FIG. 4 illustrates the six different types of decellularized ECM morsels inside their individual micro-wells in brightfield six days after seeding, and FIG. 7 illustrates decellularized morsels sections stained with H&E showing the absences of cell nuclei, and SIRIUS RED™ for the presence of fibrillar collagen (a common type of ECM) post-decellularization in all six decellularized ECM morsels.

### Spheroid-Shaped Microtissues Collected Into Tubes Then Decellularized Retained as Morsels

FIG. 5 illustrates that when fetal and adult human heart ECM spheroid microtissues were collected into one tube, decellularized, and mixed, the resulting decellularized fetal and adult human heart ECM morsels maintain their spheroid shape following the decellularization procedure without further processing.

### Decellularized Fetal Human Heart ECM Morsels Passed Through 27G Syringe 10 Times Without Further Processing FIG. 6

### Decellularized Microtissue ECM Morsels Made With Adult or Fetal Cardiac Fibroblasts Had Less Than 50 ng/mg ECM Dry Weight dsDNA

### The Mechanical Stiffness of Decellularized Microtissue ECM Morsels are Tissue and Age Specific

Mechanical stiffness (elastic moduli) of decellularized microtissue ECM morsels were measured using AFM. FIG. 10 displays the elastic moduli in kPa for three of the nine types of human decellularized microtissue ECM morsels, the range for healthy human heart tissue cited by Zile M R et al. (2015) Circulation. 2015; 131 (14):1247-59 and Guimaraes C F et al. (2020) Nature Reviews Materials. 2020; 5 (5):351-70, and the equivalent elastic modulus of injectable hydrogels that have been solidified (collagen, Matrigel, porcine cardiac ECM crosslinked with 0.1% glutaraldehyde or not) as cited in Singelyn J M et al. (2011) Macromol Biosci. 2011; 11 (6):731-8. Equivalent elastic modulus is 3 times the rheological measurements of storage moduli, assuming Poisson ratio v ˜0.5, as cited in Nemir S et al. (2010) Ann Biomed Eng. 2010; 38 (1):2-20.

### Cultured ECM is Biocompatible in Vitro

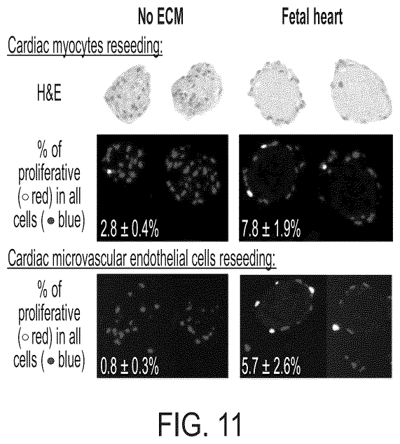

To test their biocompatibility, we decellularized and washed the fetal cardiac microtissues while still inside their individual agarose microwells (in situ decellularization). The resulting decellularized cultured human fetal heart ECM stayed within the microwells (FIG. 11). Human cardiac myocytes (HCM) and cardiac microvascular endothelial cells (HCMEC) were then seeded onto the agarose mold. The cells settled by gravity onto the cultured ECM. The cells readily adhered and survived on the ECM (FIG. 11). The cells also exhibited greater proliferation than when cultured without ECM (FIG. 11).

Proteomics was then performed to analyze the ECM composition of the decellularized ECM morsels.

### Example 3

An ECM composition as disclosed herein is used in a method of treating MI. The composition is applied in the form of an aerosol to the affected area. The heart tissue regenerates within 16 weeks.

### Example 4

An ECM composition as disclosed herein is used in a method of treating MI. The composition is applied in the form of a patch to the affected area. The heart tissue regenerates within 12 weeks.

### Example 5

An ECM composition as disclosed herein is used in a method of treating MI. The composition is applied via injection to the affected area. The heart tissue regenerates within 24 weeks.

### Example 6

An ECM composition as disclosed herein is used in a method of treating a wound. The composition is applied via injection to the affected area. The tissue regenerates within 20 weeks.

### Example 7

An ECM composition as disclosed herein is used in a method of treating a wound. The composition is applied topically to the affected area. The tissue regenerates within 32 weeks.

### Example 8

An ECM composition as disclosed herein is used in a method of treating a wound. The composition is applied in the form of a patch to the affected area. The tissue regenerates within 16 weeks.

### Example 9

An ECM composition as disclosed herein is used in a method of treating a cosmetic condition. The composition is applied via injection to the desired treatment area.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

We claim:

1. A customized flowable composition comprising decellularized human heart extracellular matrix (ECM), wherein said customized flowable composition comprises ECM comprising a desired physical or biochemical/chemical profile differing from the physical or biochemical/chemical profile of in-vivo derived adult human heart ECM in collagen and sulphated glycosaminoglycans (sGAG) content.

2. A method of producing customized decellularized human heart extracellular matrix (ECM) morsels, the method comprising:
seeding cultured human heart cells into micro-wells, wherein the cultured human heart cells generate an ECM microtissue comprised of the cultured cells and ECM produced from the cultured cells;
collecting the ECM microtissue as spheroid particles; and
decellularizing the ECM microtissue;
wherein said customized decellularized ECM morsels comprise a desired physical or biochemical/chemical profile differing from the physical or biochemical/chemical profile of in-vivo derived adult human heart ECM in collagen and sulphated glycosaminoglycans (sGAG) content.

3. The method of claim 2, wherein said decellularizing comprises mixing the microtissue with at least one of a detergent, a buffer, a DNAse, or an RNase.

4. The method of claim 2, wherein said ECM microtissue comprises spherical particles having a diameter of less than or equal to 800 μm.

5. The method of claim 2, wherein said cells comprise recombinant human cells.

6. The method of claim 2, wherein said cells comprise at least one of cardiac fibroblasts, cardiac myocytes and cardiac microvascular endothelial cells.

7. A customized flowable composition comprising decellularized human heart extracellular matrix (ECM) made by the method comprising:
seeding cultured human heart cells into micro-wells, wherein the cultured cells generate an ECM microtissue comprised of the cultured cells and an ECM;
collecting the ECM microtissue as spheroid particles; and
decellularizing the ECM microtissue;
wherein said customized flowable composition comprises ECM comprising a desired physical or biochemical/chemical profile differing from the physical or biochemical/chemical profile of in-vivo derived adult human heart ECM in collagen and sulphated glycosaminoglycans (sGAG) content.

8. The composition of claim 7, wherein said decellularizing comprises mixing the cells with at least one of a detergent, a buffer, a DNAse, or an RNase.

9. The composition of claim 8, wherein said method further comprises passing the mixture through a syringe.

10. The composition of claim 9, wherein said syringe comprises a 27 G needle.

11. The composition of claim 7, wherein said cells comprise recombinant human cells.

12. The composition of claim 7, wherein said cells comprise at least one of cardiac fibroblasts, cardiac myocytes and cardiac microvascular endothelial cells.

13. The method of claim 2, wherein said micro-wells are scaffold-free.

14. The composition of claim 7, wherein said micro-wells are scaffold-free.

15. The method of claim 2, wherein said modifying comprises adjusting at least one of cell culture media composition, culturing time, oxygen level, or the presence or amount of additional biological factors.

16. The method of claim 2, wherein said additional biological factors comprise at least one of a growth factor, a cytokine, and a drug.

17. A method of producing customized decellularized human heart extracellular matrix (ECM) morsels, the method comprising:
seeding cultured human heart cells into micro-wells, wherein the cultured cells generate an ECM microtissue comprised of the cultured cells and an ECM, wherein said ECM microtissue comprises spherical particles having a diameter of less than or equal to 800 μm;
collecting the ECM microtissue; and
decellularizing the ECM microtissue;
wherein said customized decellularized human heart ECM morsels comprise a desired physical or biochemical/chemical profile differing from the physical or biochemical/chemical profile of in-vivo derived adult human heart ECM in collagen and sulphated glycosaminoglycans (sGAG) content.

18. The customized flowable composition of claim 1, wherein the mechanical stiffness of the ECM is comparable to healthy human heart tissue.

19. The method of claim 2, wherein the mechanical stiffness of the ECM is comparable to healthy human heart tissue.

20. The customized flowable composition of claim 7, wherein the mechanical stiffness of the ECM is comparable to healthy human heart tissue.

21. The method of claim 17, wherein the mechanical stiffness of the ECM is comparable to healthy human heart tissue.
